(19) United States
(12) Patent Application Publication
(10) Pub. No.: US 2010/0147300 A1
(43) Pub. Date: Jun. 17, 2010

(54) NASAL HUMIDIFICATION AND DISPENSING DEVICE

(75) Inventors: Javier A. Lorenzati, Barcelona (ES); Justin F. Aiello, Kennebunk, ME (US)

Correspondence Address: Levenfeld Pearlstein, LLC;Intellectual Property Department, 2 North LaSalle, Suite 1300, Chicago, IL, 60602, US

(73) Assignee: CLICKMIST, A SERIES OF THE INVENTION MACHINE, LLC, Chicago, IL (US)

(21) Appl. No.: 12/336,899

(22) Filed: Dec. 17, 2008

(51) Int. Cl. A61M 15/08

(52) U.S. Cl. 128/204.14

(57) ABSTRACT

A nasal humidification and dispensing device is used to introduce a vapor into a user's nose. The device includes a pair of nostril inserts. Each nostril insert includes a base portion and an upper dome-shaped portion that, when joined, define an open interior chamber. The base portion and the dome-shaped portion have at least partially opened walls. A vapor emitting material is disposed in the interior chamber. A resilient connector extends between and connects the nostril inserts. The nostril inserts are disposed in a user's nostrils so that air that is breathed in through the inserts passes over the material and entrains vapor.

### BACKGROUND OF THE INVENTION

The present invention is directed to a nasal device. More particularly, the present invention pertains to a nasal humidification and dispensing device.

Nasal cavities are subjected to a wide variety of irritants and odors. Allergens, bacteria, viruses and the like can cause the mucosal membrane of the nasal passages to become dry, inflamed, or congested and may even lead to cracking and bleeding of the mucosal membrane. Odors may also irritate the nose and can cause stress and headaches.

It is also recognized that air in commercial airplanes and other modes of transportation (public and private) is often recirculated. Such air is typically dry, having a humidity less than comfortable for extended periods of time.

Consequently, there are numerous nasal products directed to alleviate some of these symptoms. There are nasal medications, perfumes, aromatherapy candles, and incense for odor control or mood alteration, saline solutions to alleviate nasal congestion, and other products suitable to moisten dry and irritated nasal cavities.

One known device for introducing a vapor to the nasal passages consists of a small flexible plastic bottle with a finger actuated vapor injector or pump. An elongate nozzle is inserted into the nasal passage and the pump is actuated by pulling back on a flange. The pump vaporizes and injects a fluid into the nasal passage. While this works well for discreet vapor application, it must be repeated (and possibly often) in order to be most effective.

Another device is a squeeze bottle that has a nozzle that, like the pump, fits into the nasal passage. The bottle is squeezed and vapor is driven into the nasal passage. Again, this is a discreet application and may be required to be often repeated in order to be most effective.

Accordingly, there is a need for a nasal humidification device that provides continuous application of fluid for the nasal passages. Desirably such a device enables convenient application of such fluid into the nose. More desirably such a device is disposable, comfortable and allows for repeated application of a product conveniently and effectively. Desirably still, such a device is a passive device, requiring no outward operation or motion/movement to introduce fluid into the nasal passages.

### BRIEF SUMMARY OF THE INVENTION

A nasal humidification and dispensing device is used to introduce a vapor into a user's nose. The device includes a pair of nostril inserts, each insert including a base portion and an upper dome-shaped portion that, when joined, define an open interior chamber. The base and dome-shaped portions have at least partially opened walls. For purposes of this disclosure, vapor is intended to include liquids (e.g., water) that have been vaporized, as well as solid and semi-solid materials that, when air is drawn across them, emit particulates that are carried by the air, such as solid fragrances and the like. These solid and semi-solid materials may also be materials that change state through sublimation.

A material is disposed in the interior chamber and a resilient connector extends between and connects the nostril inserts. The nostril inserts are disposed in a user's nostrils so that air that is breathed in thorough the nostril inserts becomes humidified. The material can be an absorbent material that, if a liquid is used, is saturated with the liquid, or can be formed from or in part from the solid or semi-solid material.

In a present embodiment, the upper dome-shaped portion and the base portion are formed from a plurality of spokes. The spokes of the dome-shaped portion extend from a central hub at a top of the dome-shaped portion to an open end. The hub can be formed as a disk or as an open ring. The open end is defined by a peripheral ring and defines a lip. The upper dome-shaped portion includes a lip at the ring that cooperates with a recess in the base to secure the upper dome-shaped portion to the base.

In one embodiment, the material, which if an absorbent material, can be a cellulose based material, a non-woven material or the like, is saturated. In such an embodiment, the device can include a moisture retaining storage case. The case can have a removable moisture resistant cover.

These and other features and advantages of the present invention will be readily apparent from the following detailed description, in conjunction with the claims.

### BRIEF DESCRIPTION OF THE SEVERAL VIEWS OF THE DRAWINGS

The benefits and advantages of the present invention will become more readily apparent to those of ordinary skill in the relevant art after reviewing the following detailed description and accompanying drawings, wherein:

FIG. 1 is a perspective view of an embodiment of the nasal humidification device in accordance with the principles of the present invention, the device shown in a moisture retaining container;

FIG. 2 is a top view of a device similar to that of FIG. 1;

FIG. 3 is a front view of the device;

FIG. 4 is a bottom view of the device; and

FIG. 5 is a cross-sectional view taken along line 5-5 of FIG. 2.

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiment illustrated.

It should be further understood that the title of this section of this specification, namely, “Detailed Description Of The Invention”, relates to a requirement of the United States Patent Office, and does not imply, nor should be inferred to limit the subject matter disclosed herein.

A nasal humidification device 10 dispenses a vapor into the nose (the nasal passages). The device 10 includes a pair of nostril inserts or buds 12 connected to one another by a resilient, flexible connector element 14. For purposes of this disclosure, vapor is intended to include liquids (e.g., water) that have been vaporized, as well as solid and semi-solid materials that, when air is drawn across them, emit particulates or particulate-like materials that are carried by the air, such as solid fragrances and the like. These solid and semi-solid materials may also be materials that change state through sublimation.

The buds 12 are formed as a cage-like element having a base portion 16 and an upper dome-shaped, semi-spherical shaped or semi-obround shaped containment portion 18. An internal chamber 20 is formed by the base 16 and domed 18 portions. The base 16 includes an upwardly extending collar 22 (which is short in the present embodiment) and an outwardly extending lip 24 at an upper end of the collar 22. The lip 24 and collar 22 define a recess 26.

The domed portion 18 includes an inwardly extending lip 28 at a free or open end 30 thereof. The dome lip 28 snaps over the base lip 24 and locks into the recess 26 to secure the dome-shaped portion 18 to the base 16.

The wall or walls 32 of the dome-shaped portion 18 are formed by a plurality of spokes 34 that extend from a central hub 36 at the top or peak 38 of the dome 18 to the open end 30 which is defined by a peripheral ring 40 (which is contiguous with the dome lip 28). The spokes 34 thus form multiple openings 42 in the wall 32. The hub 36 can be formed as a disk or as an open ring.

Likewise, the base 16 is formed by multiple spokes 44 disposed in a cruciform configuration to also form openings 46 in the base. In this manner, the internal chamber 20 is open to the environs through the openings 46, 42 in the dome 18 and base 16 portions. In FIG. 3, two different heights h₁₂ₐ and h_{12b} are shown, however, it will be appreciated that each device 10 will have inserts 12 of the same height h₁₂, but that inserts can be formed in a variety of different heights.

An element 48 is positioned in the internal chamber 20. The element 48, which can be an absorbent material (if, for example, a liquid is used), can be saturated with a fluid. In a present nasal humidification device 10, the fluid is water; however, those skilled in the art will appreciate that other fluids, for example, saline solution, medicaments (medicines) and the like can also be used. It will also be appreciated that fragrances, such as lavender, citrus, pine and the like can be added to the absorbent 48 or to the fluid. It will be further appreciated that solid and semi-solid materials that, when air is drawn across them, emit particulates that are carried by the air, such as solid fragrances and the like, can be used. These solid and semi-solid materials may also be materials that change state through sublimation.

One absorbent material is a cotton or cellulose based material. Other, materials, such as synthetic materials, woven or non-woven, and the like can also be used.

The resilient flexible connector element 14 is formed to accommodate a sight spreading or contraction of the distance between inserts 12 to accommodate noses of different sizes.

Alternately (not shown), a capsule or other liquid-containing member can be disposed in the interior chamber along with, for example, a saturatable material. The capsule can be pierced (for example by a cleave located on the inside of the base (not shown) or other piercing means to release the liquid from the capsule and to saturate the material. The capsule can be formed from, for example, a gelatin or gelatin-like material.

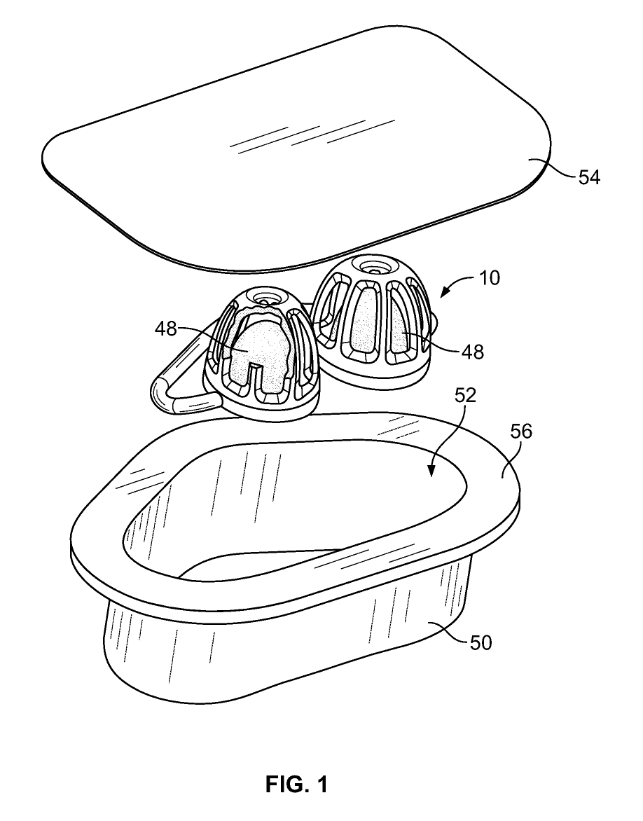

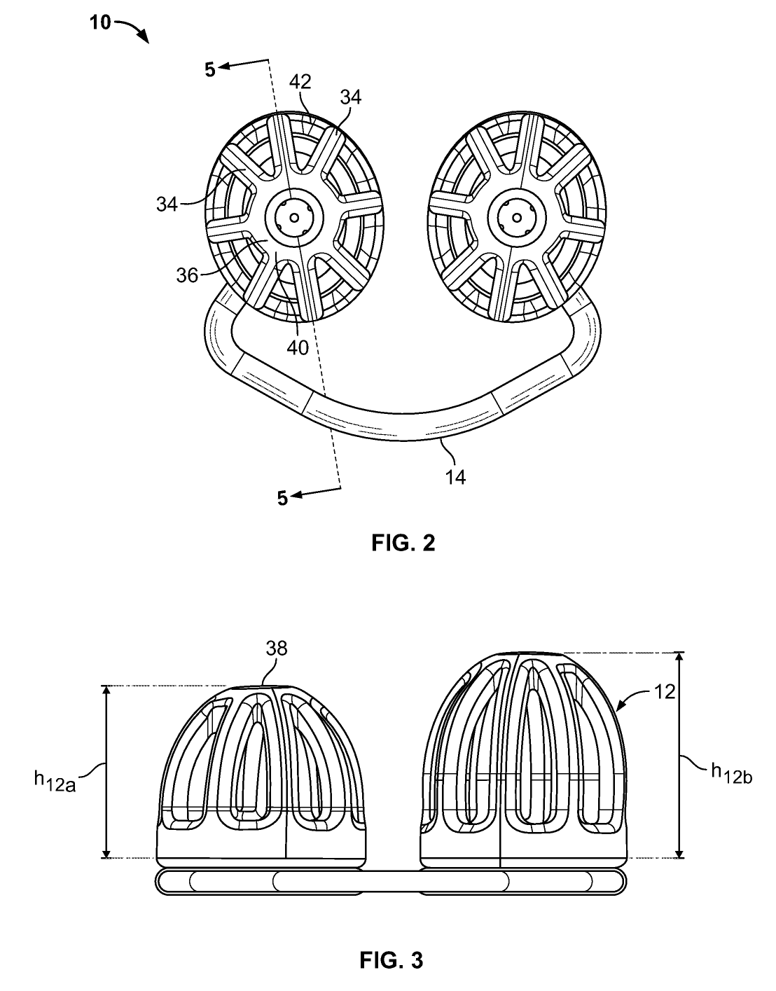

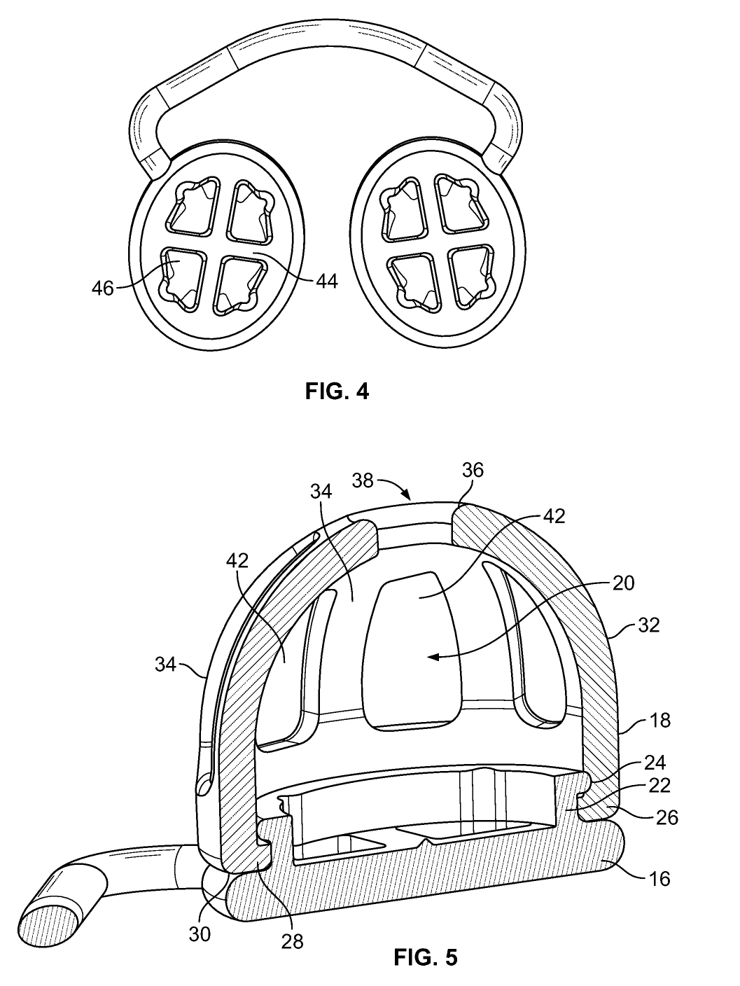

A present nasal humidification device 10, particularly the type having a pre-saturated material 48, is preferably stored in a sealed, moisture-retaining container 50. One exemplary container 50 is illustrated in FIG. 1 and has a shape that is complementary to the nasal humidification device 10. The container 50 has an open top 52 that allows easy access to the device 10 and includes a moisture retaining seal 54, such as a foil seal, disposed over the top 52 of the container 50. The container top 52 can include an outwardly extending flange 54 to provide a larger area over which the seal 54 can be affixed to the container 50. Other packaging configurations (such as multi-packs and the like) are also contemplated.

In use, the device 10 is removed from the container 50 and the buds 12 are inserted into the user's nostrils. Breathing through the buds 12 draws air from the environs, through the element 48 to entrain vapor in the air. In the case of a saturated (e.g., water saturated with or without an additive) element, to humidify air as it is breathed in.

All patents referred to herein, are incorporated herein by reference, whether or not specifically done so within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

What is claimed is:

1. A nasal humidification and dispensing device used to a introduce a vapor into a user's nose comprising:
a pair of nostril inserts, each nostril insert including a base portion and an upper dome-shaped portion that, when joined, define an open interior chamber, the base portion and the dome-shaped portion having at least partially opened walls;
a vapor emitting material disposed in the interior chamber; and
a resilient connector extending between and connecting the nostril inserts,
wherein the nostril inserts are disposed in the user's nostrils so that air that is breathed in through the nostril inserts passes over the material and entrains vapor.

2. The nasal humidification and dispensing device in accordance with claim 1, wherein the upper dome-shaped portion is formed from a plurality of spokes that extend from a central hub at a top of the dome-shaped portion to an open end.

3. The nasal humidification and dispensing device in accordance with claim 1, wherein the open end is defined by a peripheral ring.

4. The nasal humidification and dispensing device in accordance with claim 3, wherein the peripheral ring defines a lip.

5. The nasal humidification and dispensing device in accordance with claim 2, wherein the hub can be formed as a disk or as an open ring.

6. The nasal humidification and dispensing device in accordance with claim 1 wherein the base portion is formed from a plurality of spokes.

7. The nasal humidification and dispensing device in accordance with claim 1 wherein the vapor emitting material is an absorbent material.

8. The nasal humidification and dispensing device in accordance with claim 7 wherein the absorbent material is saturated with a fluid.

9. The nasal humidification and dispensing device in accordance with claim 8 wherein the fluid is water.

10. The nasal humidification and dispensing device in accordance with claim 8 wherein the fluid contains an additive.

11. The nasal humidification and dispensing device in accordance with claim 8 wherein the fluid contains a medicament.

12. The nasal humidification and dispensing device in accordance with claim 7 wherein the absorbent material is a cellulose based material.

13. The nasal humidification and dispensing device in accordance with claim 7 wherein the absorbent material is a non-woven material.

14. The nasal humidification and dispensing device in accordance with claim 1 including a moisture retaining storage case.

15. The nasal humidification and dispensing device in accordance with claim 14 wherein the case has a removable moisture resistant cover.

16. The nasal humidification and dispensing device in accordance with claim 1 wherein the vapor emitting material is a solid or semi-solid material that, when air is drawn thereacross, emits particulate or particulate-like material that is carried by the air.
